(19) United States
(12) Patent Application Publication
(10) Pub. No.: US 2009/0062645 A1
(43) Pub. Date: Mar. 5, 2009

(54) METHOD FOR DIAGNOSIS AND TREATMENT OF PROSTATE CANCER

(76) Inventors: Jens Fehre, Hausen (DE); Ralf Nanke, Neunkirchen am Brand (DE); Martin Stetter, Muenchen (DE)

Correspondence Address: SCHIFF HARDIN, LLP;PATENT DEPARTMENT, 6600 SEARS TOWER, CHICAGO, IL, 60606-6473, US

(21) Appl. No.: 12/203,198

(22) Filed: Sep. 3, 2008

(30) Foreign Application Priority Data
Sep. 3, 2007 (DE) 10 2007 041 833.9

(51) Int. Cl. A61B 8/13; A61B 5/00

(52) U.S. Cl. 600/437; 600/300

(57) ABSTRACT

In a method for diagnosis and treatment of a patient with a tumor relating to prostate cancer, the following steps are implemented. A differential diagnosis of prostate cancer versus prostatitis and/or BPH is conducted on a patient using a cost-effective diagnosis method. If prostate cancer is diagnosed in the patient using a cost-effective measurement method, a characteristic value for the tumor aggressiveness of the prostate cancer is determined. A watchful waiting treatment is implemented with the patient given a characteristic value below a predeterminable first limit value. The size and position of the tumor is determined using a cost-effective method given a characteristic value above the first limit value. A cost-effective ultrasonic theranosis or a conventional therapy is conducted for a size below a second predeterminable limit value. The presence of metastases in the patient is checked, with a cost-intensive method generating image information, for a size above the second limit value. A metastasis treatment is implemented in the event that metastases are present. In the event that no metastases are present, a tumor treatment based on the aforementioned image information generated is implemented.

### BACKGROUND OF THE INVENTION

1. Field of the Invention

The invention concerns a method for diagnosis and treatment of a patient with regard to prostate cancer, wherein the prostate cancer is accompanied by a tumor.

2. Description of the Prior Art

A goal in everyday medical routine is to examine a patient for prostate cancer, i.e. to generate a diagnosis and to implement treatment in the event of a positive diagnosis with regard to prostate cancer.

A concentration of the prostate-specific antigen (PSA test) is initially conducted in a known, clinically established workflow for diagnosis and treatment of prostate cancer. A transrectal ultrasound examination is conducted if an abnormality is present. Given a corroborated suspicion of prostate cancer, one or more biopsies are conducted. A microscopic morphological examination supplies what is known as the Gleason degree, which is a measurement of the aggressiveness of the tumor. A method for targeted diagnosis of prostate cancer (PCa) in the early stage or pre-stage (“high grade Prostatic Intraepithelial Neoplasia”—hgPIN) is not known. Therapy for prostate cancer in later stages is subsequently conducted with one of the following methods.

A significant percentage of PCa tumors grow very slowly and are therefore harmless, if anything. This is often not realized and the patient is incorrectly treated. Nevertheless, if this is realized a treatment is foregone and the tumor behavior is tracked at regular examinations. This active monitoring is also called “watchful waiting”.

Moreover, it is known to operate on the patient or to possibly subject the patient to a cryo-treatment. If the cancer is limited to the prostate, the prostate (and thus the cancer as well) can be completely removed. This method is designated as radical prostatectomy. It is accompanied by significant burdens for the patient, does not promise a 100% chance of recovery and may be unnecessary due to misdiagnosis, namely an undetected, slowly-growing tumor or prostatitis.

It is known to subject the patient to radiation therapy or also brachytherapy. Tumor tissue can thereby be locally killed. However, this also leads to negative effects on the adjacent tissue, for example the bladder or the intestine.

Given treatment with HIFU (high intensity focused ultrasound), the tumor tissue fired upon is selectively, externally heated and destroyed. The tumor position and boundary (thus the perimeter of the tumor) must thereby be precisely known. This information can be supplied via simultaneous MRT. The resulting procedure is complicated.

A hormone treatment is primarily implemented in the early stage. Testosterone induces tumor growth and is hereby suppressed.

In immune therapy the immune system is prompted to help itself.

Chemotherapy (i.e. a generic administration) is primarily implemented given advanced cancer.

The known methods exhibit serious drawbacks, such that the diagnosis and treatment of prostate cancer is limited in efficiency.

The known, clinically established, non-invasive methods can only imprecisely diagnose prostate cancer and distinguish it from prostatitis and BPH. An incorrect treatment of incorrectly diagnosed prostatitis or BPH results, namely a cancer therapy.

The problem is additionally exists that a high percentage of prostate tumors are less aggressive but a small percentage of tumors are extremely aggressive, which entails a poor possibility for prognosis of the course of disease. The determination of a tumor grade (i.e. of a numerical characteristic value for its aggressiveness) is therefore very highly relevant for therapy planning with the patient. For this differentiation (i.e. the determination of the tumor grade) it is known only to determine the characteristic value with the use of (often multiple) painful biopsies, thus with significant physical and mental stress of the patient. Since the tumor grade is frequently incorrectly determined, a high percentage of over-therapy of less aggressive tumors results, as well as a dangerous non-detection of aggressive tumors. Due to the cited disadvantages, a sub-optimal care of patients results.

Novel screening, diagnosis, therapy planning and treatment methods exist or, respectively, are in development that can expect greater diagnostic precision and therapeutic impact on the basis of molecular mechanisms.

A more cost-effective molecular screening test for differential diagnosis of prostate cancer in contrast to prostatitis and benign prostate enlargement (BPH—benign prostate hyperplasia), for example with near-infrared measurement, is thus described in the German Patent Application 10 2007 028 659.9, filed Jun. 21, 2007.

A non-invasive grading, thus a determination of the characteristic value of the tumor aggressiveness with the aid of a near-infrared arrangement is described in the German Patent Application 10 2007 037 008.5, filed Aug. 6, 2007.

Furthermore, a method for magnetic resonance imaging with ferromagnetic, molecularly marked particles as a contrast agent in order to precisely determine the shape and size of the primary tumor is described in U.S. patent application having Atty. Docket No. P08,0241, filed simultaneously herewith, which has the same priority as the present application. If an angiogenesis marker (for example VEGF or alpha(v) beta(3) integrin marker) is used, metastases in the entire body can hereby also be detected.

A method for ultrasound theranostics is also described in U.S. patent application having Atty. Docket No. P08,0245, filed simultaneously herewith, which has the same priority as the present application. Molecular markers and medicine transporters are hereby simultaneously administered to the patient. The marker indicates the extent of the prostate tumor; the therapeutic agent is, if applicable, released on site via ultrasonic exposure.

The cited methods have varying properties with regard to precision, diagnostic bandwidth, therapeutic impact and costs. A perfect care of every patient would be ensured if all cited methods were implemented on all patients. However, this is unrealistic due to the health care cost explosion that would be triggered by such an approach.

### SUMMARY OF THE INVENTION

An object of the present invention is to provide a procedure for diagnosis and treatment of prostate cancer in a patient, which ensures the optimal usage of the cited and similar innovative methods for each patient.

The method according to the invention is a procedure that uses the novel, molecularly supported diagnosis and treatment techniques described above optimally with regard to effectiveness, and simultaneously avoids unnecessary or inefficient steps.

The object is achieved by a method for diagnosis and treatment of a patient with regard to prostate cancer, with the following steps:

a) a differential diagnosis of prostate cancer versus prostatitis and/or BPH is conducted on a patient using a cost-effective diagnosis method,
b) if prostate cancer is diagnosed in the patient using a cost-effective measurement method, a characteristic value for the tumor aggressiveness of the prostate cancer is determined,
c) a watchful waiting treatment is implemented with the patient given a characteristic value below a predeterminable first limit value,
d) the size and position of the tumor is determined using a cost-effective method given a characteristic value above the first limit value,
e) a cost-effective ultrasonic theranosis or a conventional therapy is conducted for a size below a second predeterminable limit value,
f) the presence of metastases in the patient is checked, with a cost-intensive method generating image information, for a size above the second predeterminable limit value,
g) a metastasis treatment is implemented in the event that metastases are present, and
h) in the event that no metastases are present, a tumor treatment based on the image information generated in Step f) is implemented.

Every prostate cancer patient receives the optimal assessment, diagnosis and treatment with simultaneously reduced costs via the method according to the invention. For a given patient, the efficiency of the care is therefore maximized and this occurs simultaneously with minimized diagnosis or, respectively, treatment costs.

The novel medical workflow specified by the method allows novel molecularly supported screening, diagnosis and therapy methods to be used so that the patient care is the best possible but unnecessary steps are consistently omitted. The efficiency of the care of prostate cancer can thus be increased. Higher care quality results with simultaneously reduced costs, which is of particularly great importance in light of the demographic change and the fact that prostate complaints predominantly affect older men.

Due to the more precise coordinate and higher impact of the treatment, treatment durations are minimized, recovery rates are increased and side effects are minimized.

Due to the better and more precise information of the patient, but also due to the omission of unnecessary diagnostic steps and the non-invasive nature of the diagnostic or, respectively, therapeutic methods, the patients are exposed to fewer mental and other stresses.

An additional advantage is that the identical or very similar molecular markers (thus identical target molecules) can be used in part in the various steps.

Instead of the cost-effective method in Step d), the cost-effective method from Step f) can also be implemented to determine the size and position of the tumor and to check for the presence of metastases and to generate the image information. Although the method is more expensive, it delivers more precise information.

The patient can additionally be tested for concentrated or distributed metastases in Step g). A chemotherapy is implemented in the event of distributed metastases and a resection in the event of concentrated metastases.

In the event of concentrated metastases, a chemotherapy can still additionally be implemented after the resection.

### BRIEF DESCRIPTION OF THE DRAWINGS

The single FIGURE is a flowchart of an embodiment of a method according to the invention to diagnose and treat prostate cancer in a patient.

### DESCRIPTION OF THE PREFERRED EMBODIMENTS

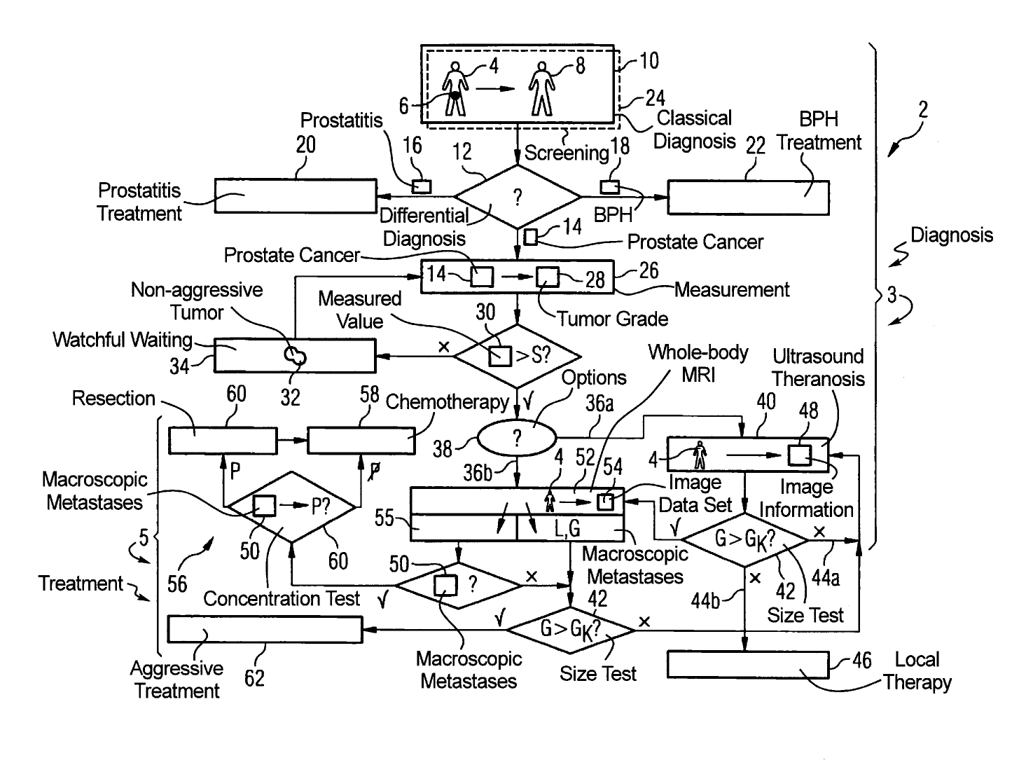

FIG. 1 shows the medical workflow 2 that can essentially be sub-divided into diagnosis 3 and treatment 5, wherein both procedures intermix in part. The care process of a patient 4 begins by the patient visiting physician 8. This physician 8 conducts a classical diagnosis 10 on the patient 4. Symptoms or abnormalities in the prostate 6 of the patient 4 are determined. These abnormalities can be, for example, be an increased PSA level. The patient 4 is examined further, for example, using a palpation, a transrectal ultrasound examination and/or biopsies with subsequent histology.

The goal of this classical diagnosis 10 is a differential diagnosis 12 in which the finding of prostate cancer 14 is distinguished from prostatitis 16 or BPH 18. If prostatitis 16 is diagnosed, a workflow to the prostatitis treatment 20 is initiated (not explained in detail herewith). If the assessment is without pathological findings, no illness exists and the patient 4 does not need to be treated further. In the case of BPH, an uncomplicated BPH treatment 22 (not explained in detail) is implemented.

As an alternative to the classical diagnosis 10, abnormalities can also be detected, for example, in the framework of a broadly applied screening test 24 of the elderly male population. Due to the large number of tests, a particularly cost-effective method can be used in the screening that in particular can provide a yes/no conclusion about the presence of prostate cancer 14 with high sensitivity and specificity, and in particular can distinguish this from prostatitis 16 and BPH 18. Examples of such methods exist in the molecular, contrast-enhanced ultrasound examination or the molecular near-infrared examination as explained above.

In the event of prostate cancer 14, the most important information linked with this is the tumor aggressiveness, since this is crucial for the further therapy requirements of the patient 4. In a next step, the tumor grade 28 is therefore determined as a characteristic value in a measurement method 26. In the described exemplary embodiment, this occurs non-invasively with a molecularly targeted near-infrared examination. This is non-imaging and is designed as a cost-effective, transrectal examination. A measured value 30 that is proportional to the Gleason grade is yielded as a result of the examination. If this number does not lie above a threshold S, a non-aggressive tumor 32 is diagnosed.

As a result the patient with a non-aggressive tumor 32 is given over to a “watchful waiting” monitoring 34. The aggressiveness of the tumor is measured there at regular intervals (the measured value 30 is thus determined in the measurement method 26). Optionally, the size G of the tumor 32 can also be regularly measured in addition (not shown in FIG. 1), for example via molecularly targeted ultrasound diagnosis.

If the tumor 32 is diagnosed as aggressive in the measurement method 26 (thus the measured value 30 is greater than S), an additional need for clarification results with regard to the progression of the illness. The position L and size G of the tumor 32 in the patient 4 are therefore to be determined precisely, and the presence of metastases 50 are possibly to be checked. This is also called “staging”.

Two options 36a and 36b thus exist at this point in time at the branch 38: Option 36a is a more cost-effective method that is, however, limited to the prostate 6 and consists of imaging the tumor 32 according to position L and size G, i.e. the acquisition of image information 48 and its simultaneous treatment with a prostate cancer ultrasound theranosis 40. The size G of the tumor 32 is checked in a size test 42. If the tumor falls below a size predetermined by the physician in the form of limit value GK, two options 44a,b again exist. In option 44a, the tumor 32 can be treated via repeated application of ultrasound theranosis 40. A course monitoring of the recovery of the patient 4 is hereby simultaneously possible due to the image information 48.

A local therapy 46 of smaller primary tumors is supplied to the patient 4 in option 44b, wherein the image information 48 about position L, shape and size G (acquired from the ultrasound theranosis 40) directly influence the therapy planning, however. Known therapies 46 are, for example, HIFU, brachytherapy, x-ray exposure, etc.

If a tumor size (thus size G) above the threshold GK is established in the size test 42, an increased danger exists that the illness has already progressed far and metastases 50 have occurred. An affected patient 4 will therefore now undergo a whole-body MRI 52 with molecular angiogenesis markers, which delivers an image data set 54. This examination is relatively expensive but—in addition to the more precise position L, shape and size determination G of the primary tumor 52—primarily also allows the detection of possibly present macroscopic metastases 50 in the entire body of the patient 4 in a metastasis test 55. However, this examination is applied only for patients 4 with clear indication, which contributes to the efficiency increase of the medical care in workflow 2.

As a second option 36b, given a diagnosed aggressive tumor 32 the MRI examination 52 can also be directly skipped to as an alternative to ultrasound theranosis 40.

If metastases 50 are detected in the framework of the MRI 52, an expanded treatment 56 of the patient 4 beyond the prostate 6 is required. For example, a chemotherapy is initiated in addition to the removal of the prostate tumor 32 or, respectively, the entire prostate 6.

In a concentration test 60 it is therefore checked whether the metastases 50 are concentrated. Namely if the metastases 50 are concentrated (thus exist in a small body region P), these can possibly also be operatively removed in a resection 61 before the chemotherapy 58 is initiated.

If no metastases 50 are present as a result of the MRI or the metastasis test 55, the patient 4 suffers only from the primary tumor 32. Depending on the selected option 36a,b of the diagnostic workflow at the branch 38, the tumor 32 can now be large or small; an additional size test 42 is therefore implemented. However, in each case its position (thus position L and size G) is precisely known.

If the tumor 32 is large (thus G>GK), an aggressive treatment 62 (such as, for example, resection, exposure and/or HIFU) can be implemented supported by the image information from the image data set 54.

If the tumor is small (thus G<GK), theranosis 40 and/or therapy 46 is supplied to the patient 4.

Although modifications and changes may be suggested by those skilled in the art, it is the intention of the inventors to embody within the patent warranted hereon all changes and modifications as reasonably and properly come within the scope of his contribution to the art.

We claim as our invention:

1. A method for diagnosis and treatment of a patient having a tumor associated with prostate cancer, comprising the steps of:
(a) implementing a differential diagnosis with regard to the patient using an economical diagnosis method to differentiate an occurrence of prostate cancer from prostatitis and/or BPH;
(b) if prostate cancer is diagnosed in said patient, using an economical measurement method to identify a characteristic value representing a tumor aggressiveness of the prostate cancer;
(c) implementing a watchful weighting treatment with regard to the patient if said characteristic value is below a predetermined first value;
(d) determining a size and position of the tumor using an economical method if said characteristic value is above said first value;
(e) implementing an economical ultrasound theranosis or another therapy if said size of said tumor is below a second value;
(f) detecting a presence of metastases in said patient with an economical method to generate image information if said size is above said second value;
(g) implementing a metastases treatment if metastases are detected in step (f); and
(h) if no metastases are detected in step (f), implementing a tumor treatment based on the image information generated in step (f).

2. A method as claimed in claim 1 comprising, as said economical method in step (d), implementing said economical method from step (f) to determine the size and position of the tumor and to check for the presence of metastases and to generate said image information.

3. A method as claimed in claim 1 comprising additionally testing said patient for concentrated or distributed metastases in step (g) and implementing a chemotherapy if distributed metastases are detected and implementing a resection if concentrated metastases are detected.

4. A method as claimed in claim 3 comprising implementing a chemotherapy after said resection.
